(19) United States
(12) Patent Application Publication
(10) Pub. No.: US 2015/0212059 A1
(43) Pub. Date: Jul. 30, 2015

(54) METHOD AND DEVICE FOR DETECTION OF HEAVY METALS IN WATER USING DYE NANO-COMPLEXANTS AND A POLYMERIC FILM

(71) Applicant: Technion Research & Development Foundation Ltd., Haifa (IL)

(72) Inventors: Israel SCHECHTER, Haifa (IL); Bella DOLGIN, Beer Sheva (IL)

(21) Appl. No.: 14/606,307

(22) Filed: Jan. 27, 2015

Related U.S. Application Data
(60) Provisional application No. 61/931,722, filed on Jan. 27, 2014.

(51) Int. Cl. G01N 33/18; G01N 21/78; G01N 21/25

(52) U.S. Cl. CPC G01N 33/1813; G01N 21/25; G01N 21/78

(57) ABSTRACT

The present invention relates to a kit, device and method for detection of metals in aqueous media. The invention is based on complexation reactions of organic azo-dyes with heavy metals, utilizing a specially designed polymeric film matrix to which an organic azo-dye and an organic solvent are added. When submerged in water contaminated with heavy metals, the polymeric film changes its color. The azo-dyes are injected into the tested water, resulting in formation of nano-particles of insoluble complexes. The polymeric film embeds and dissolves these nano-particles and thus allows for spectral and/or visual analysis.

### CROSS REFERENCE

This application claims priority to U.S. Provisional Patent Application No. 61/931,722 filed Jan. 27, 2014, incorporated by reference herein in its entirety.

### FIELD OF THE INVENTION

The present invention relates to a device, kit and method for detection of metals in aqueous media, based on embedding dyes in a polymeric matrix skeleton, together with an organic solvent.

### BACKGROUND OF THE INVENTION

The toxicity of heavy metal ions is high, and the maximum allowed concentrations in drinking water are in the parts-per-billion (ppb) range (e.g., 50 ppb for Ni, 5 ppb for Cd and 110 ppb for Co). Controlling the concentrations of heavy metals in water is also needed in semiconductor and some other industries. The most routinely used methods for such analyses are furnace atomic absorption spectroscopy and inductively coupled plasma atomic emission spectroscopy. Other methods include dispersive X-ray fluorescence, UV-VIS spectroscopy and differential pulse cathodic stripping voltammetry. These are costly and labor intensive technologies and simpler and lower-cost alternatives are preferred. Small electrochemical sensors and sensor arrays for heavy metals have been also suggested, however, their current performance is rather poor for water analysis. Nanomaterials are interesting alternative components to be integrated into various electrochemical devices for heavy metals detection. However, thus far these devices suffer from issues related to stability and reproducibility as well as aspects associated to the toxicity of the used materials.

A different approach is based on creating complexes of heavy metals and detecting their optical properties. An example of such a method was based on visual detection using dye-loaded membranes [1]. Several ions, including Hg⁺², Ag⁺, Cu⁺², Ni⁺², Pd⁺², Zn⁺², Co⁺³ and Fe⁺² were detected using various aromatic dyes and the detection limits were in the sub-ppm range.

Azo compounds are well known aromatic dyes that have the ability to form colored complexes with heavy metals [2-5]. In particular, 1-(2-thiazolylazo)-2-naphthol (TAN) and (1-(2-pyridylazo)-2-naphthol (PAN) were widely used for spectral analysis of Pd⁺², Co⁺², UO₂⁺², Cu⁺² and other rare earth metals [6-9] and for their extraction and separation[10-15]. There were also used as chelating reagents in flame atomic absorption spectrometry (FAAS) analysis [16].

Lowering detection limits, without using complicated analytical instruments, should involve pre-concentration steps. Recently, three filter concentration techniques have been examined and compared: (a) Depositing the complexant on a filter and immersing it into the water was the simplest procedure. However, this method suffers of several drawbacks, including high sensitivity to local concentration gradients, which affect the filter coloration. (b) Performing the complexation in a vessel and filtering the products provides good results, but requires laboratory work. (c) Depositing the complexant particulates on a filter and passing the tested solution through the filter. This method provides good calibration plots, however, it is very sensitive to individual experimental conditions, such as filtering flow and local geometric parameters.

In addition to filter concentration methods, other approaches for fixation of indicator molecules to different support media have also been suggested. These include PVC based liquid membranes, immobilization to chelating resin and to impregnated resin, application of polyurethane foam and immobilization on to a C18 bonded silica support. Test strips were also successfully produced by filtrating organic reagents through cellulose ester membranes. Test strips based on immunochromatographic assay have also been developed, with detection limits of ca. 80 ppb for Cr ions. Drawbacks include complicated synthetic procedures, insufficient sensitivity, requirements for auxiliary additives, and difficulty in controlling the concentration of the reagent and its uniformity.

There remains an unmet need for efficient methods for determination and/or quantification of low concentrations of heavy metals in solutions and other media, that overcomes the deficiencies of the prior art.

### SUMMARY OF THE INVENTION

The present invention relates to a device, kit and method for determination of presence and/or amount of metals in aqueous media.

As contemplated herein, the present invention provides an efficient system and method for producing test strips for determination of low concentrations of heavy metals in aqueous media, based on embedding an organic dye in a polymeric matrix skeleton, together with an organic solvent. The organic solvent is used to modify the properties of the solid film, such that it can extract and accommodate the organic metal-complex from the water. Detection of metals is achieved by contacting the polymeric film with an aqueous medium containing the organic dye, followed by visual inspection and/or spectral analysis of the polymeric film. The organic dye is added to the aqueous medium, as a means of assisting and/or enhancing the sensitivity of the color change in the polymeric film.

In view of the complexity of the prior art methods, such a simple strip test possesses significant advantages.

Thus, in one aspect, the present invention relates to a method for detection of metals in an aqueous medium suspected of comprising metals, the method comprising the steps of: (a) contacting a test aqueous medium comprising an organic dye with a polymeric film comprising an organic solvent and the organic dye; and (b) detecting the presence of metals in the aqueous medium by visual inspection and/or by spectral analysis of the polymeric film. The organic dye may be added to the test aqueous medium prior to or simultaneously with the step of contacting the aqueous medium with the polymeric film.

According to another aspect, the present invention relates to a polymeric film device for detection of metals in an aqueous medium, the device comprising a polymeric film comprising an organic solvent and an organic dye. In a currently preferred embodiment, the polymeric film is selected from polyvinyl chloride (PVC), polyvinyl alcohol (PVA) and combinations and copolymers thereof; the organic solvent is Bis(2-ethylhexyl)phthalate, and the organic dye is an azo dye selected from the group consisting of 1-(2-thiazolylazo)-2-naphthol (TAN), (1-(2-pyridylazo)-2-naphthol (PAN), and combinations thereof. According to another aspect, the present invention relates to a kit for detection of metals in an aqueous medium, the kit comprising (a) a polymeric film comprising an organic solvent and an organic dye; and (b) a composition comprising the organic dye; wherein the presence of metals is detected by adding the organic dye composition to the aqueous medium followed by contacting the aqueous medium with the polymeric film, and detecting the presence of metals in the aqueous medium by visual inspection and/or by spectral analysis of the polymeric film.

According to another aspect, the present invention relates to a system for detecting metals in an aqueous medium, the system comprising (a) a polymeric film comprising an organic solvent and an organic dye; and (b) an aqueous medium comprising the organic dye; wherein the presence of metals is detected by contacting the aqueous medium with the polymeric film, and detecting the presence of metals in the aqueous medium by visual inspection and/or by spectral analysis of the polymeric film.

According to the principles of the presence invention, the organic dye complexes with the metals present in the aqueous media, so as to form a metal-dye complex. The presence and/or amount of metals can then be detected by subjecting the organic film to spectral analysis and/or visual inspection as described herein. Without wishing to be bound by any particular theory or mechanism of action, it is contemplated that the metal-dye complex may be formed by multiple mechanisms. In one embodiment, the metal-dye complex is formed in the aqueous medium and is then absorbed/dissolved into the polymeric film. In another embodiment, the metal-dye complex is formed in the film, for example by metal ions diffusing into the film and complexing with the dye either inside or on the surface of the film. Each possibility represents a separate embodiment of the present invention.

In one preferred embodiment, the polymeric film is provided in the form of a test strip, which may be submerged (completely or partially) into the aqueous medium. The test strip may then be subjected to spectral analysis and/or visual inspection as described herein.

In one embodiment, the detection step (b) comprises comparing the color of the polymeric film before and after contact with the aqueous medium, wherein a change in the color of the polymeric film is indicative of the presence of metals in the aqueous medium.

In another embodiment, the detection step (b) comprises spectral analysis of the polymeric film. Spectral analysis may be performed by any means known to a person of skill in the art. In one preferred embodiment, spectral analysis comprises comparing the reflectance spectra of the polymeric film before and after contact with the aqueous medium, wherein a change in reflectance spectra of the polymeric film is indicative of the presence of metals in the aqueous medium Advantageously, the measuring step may be performed at a wavelength at which the dye-metal complex reflects, and wherein the dye has a small or no reflectance.

According to one embodiment, the polymeric film is polyvinyl chloride (PVC)/polyvinyl alcohol (PVA).

According to another embodiment, the organic dye is an azo dye. Preferably, the azo dye is selected from the group consisting of 1-(2-thiazolylazo)-2-naphthol (TAN) and (1-(2-pyridylazo)-2-naphthol (PAN). Each possibility represents a separate embodiment of the present invention.

According to another embodiment, the organic solvent is Bis(2-ethylhexyl)phthalate.

According to another embodiment, the aqueous medium comprises heavy metals selected from the group consisting of cadmium (Cd), nickel (Ni), cobalt (Co), copper (Cu), their ionized forms, including salts and oxides, and combinations thereof.

The polymeric film used in the present invention may be prepared by any method known to a person of skill in the art. In one preferred embodiment, the polymeric film is prepared by polymerizing monomers in the presence of the organic solvent and the organic dye.

According to the principles of the present invention, the organic dye is added both to the polymeric film and the aqueous medium. To aid its dissolution, the organic dye is preferably dissolved in an organic solvent (preferably a water-miscible organic solvent such as acetone and the like), prior to addition to the aqueous medium.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Reflectance spectra of the polymeric films containing PAN (85 nmole cm⁻²) and TAN (100 nmole cm⁻²), before their exposure to heavy metals.

FIG. 2: The effect of the initial concentration of TAN in the polymeric film upon the reflectance of Ni-TAN complex. Ni ion concentration in the water and the accumulation times were kept constant.

FIG. 3: Reflectance spectra of the polymeric film in the range corresponding to the Ni-TAN complex, for a series of Ni ion concentrations. (a) Trace amount of TAN was injected into the water (0.04 μM). (b) A significant amount of TAN was injected (1.2 μM).

FIG. 4: (a) Reflectance spectra of Ni-TAN complex formation and accumulation in the polymeric film, as a function of time, after exposure to Ni ions. (b) Photo of the polymeric film at various times after exposure to 2.5 ppm Ni ions. The color development shows the Ni-TAN complex formation and accumulation in the polymeric film.

FIG. 5: The slope of the calibration plot for Ni ions in water, measured at 598 nm as a function of the concentration of the TAN added to the solution. The range of Ni ion concentration in the calibration plots was 0.5-5 ppm.

FIG. 6: Schematic representation of the TAN (a) and PAN (b) species at various pH ranges.

FIG. 7: Reflectance intensity of polymeric films that were immersed in 2.5 ppm nickel (a) or cadmium (b) ion solutions for 70 min. (PAN solution concentration: 1.2 μM. PAN film concentration: 1.0 μmole cm⁻²).

FIG. 8: Calibration plot for analysis of Ni ions in water, using PAN (a) and TAN (b) embedded polymeric film.

FIG. 9: Photos of polymeric films exposed to several concentrations of Ni ions.

FIG. 10: (a) Reflectance spectra the PAN embedded polymeric film, when exposed to a series of Cd ion concentrations. (b) The resulted calibration plot, which indicates a LOD of ca. 77 ppb.

FIG. 11: Reflectance spectra and the resulted calibration plots for the PAN (a,b) and TAN (c,d) embedded polymeric films, when exposed to a series of Co ion concentrations. The calculated LODs are of ca. 120 ppb (for PAN) and 110 ppb (for TAN).

### DETAILED DESCRIPTION OF THE PRESENT INVENTION

The present invention relates to a kit, device and method for detection of metals in aqueous media. As contemplated herein, an optical analytical method, based on complexation reactions of organic azo-dyes with heavy metals, is proposed for the first time. The invention is based on a specially designed polymeric film matrix to which an organic azo-dye and an organic solvent are added. When submerged in water contaminated with heavy metals, the polymeric film changes its color. The azo-dyes are injected into the tested water, resulting in formation of nano-particles of insoluble complexes. The polymeric film embeds and dissolves these nano-particles and thus allows for spectral and/or visual analysis to detect the presence and/or amount of metals present. In some embodiment, the film comprises a PVC/PVA polymeric skeleton and an organic solvent, e.g., Bis(2-ethylhexyl)phthalate, which possesses high affinity to the heavy metal nano-complexes. The method has been exemplified herein for Cd, Ni and Co ions, but is applicable to the detection of a wide variety of metals in water and other aqueous containing media.

The method of the present invention is sensitive in the sub parts-per-million (ppm) range, and is advantageously performed by submerging (e.g., dipping) the polymeric film in the form of a test strip to a test aqueous media comprising heavy metals, to which the organic dye has been added.

The term “aqueous media”, or “aqueous medium”, as used herein, refers an aqueous solution, dispersion or suspension, which may contain metals to be detected by the methods and kits of the present invention.

Any organic dye may be used in the methods and kit of the present invention. According to some preferred embodiments, the organic dye is an azo dye. An Azo dye is a compound bearing the functional group R—N═N—R′, in which R and R′ are aryl. In some embodiments, the azo dye is selected from pyridylazo derivatives, thiazolylazo derivatives, benzothiazolylazo derivatives and quinolylazo derivatives. Preferably, the azo dye is selected from the group consisting of 1-(2-thiazolylazo)-2-naphthol (TAN) and (1-(2-pyridylazo)-2-naphthol (PAN). Other azo-based dyes that may be used in the context of the present invention include, but are not limited to, 4-(2-Pyridylazo)resorcinol (PAR), 1-(2-benzothiazolylazo)-2-naphthol (BTAN), 2-(8-quinolylazo)-4,5-(diphenyl)imidazole (QAI), and 2-(5-bromo-2-pyridylazo)-5-(diethylamino)phenol (BrPADAP). Additional dyes that can be used in the context of the present invention include, but are not limited to, acridine dyes, anthaquinone dyes, arylmethane dyes (including diarylmethane and triarylmethane dyes), diazonium dyes, nitro dyes, nitroso dyes, phthalocyanine dyes, quinone-imine dyes, azin dyes (e.g., Eurhodin dyes, safranin dyes), indamin dyes, indophenol dyes, oxazin dyes, oxazone dyes, thiazine dyes, thiazole dyes, xanthene dyes, fluorene dyes, pyronin dyes, fluorone dyes, rhodamine dyes, and the like. Each possibility represents a separate embodiment of the present invention.

Dye concentrations to be used in the methods of the present invention can be determined by a person of skill in the art, and will depend on the particular dye being used and the metal(s) being detected. Suitable concentrations range from 0.1 to about 10 μM, e.g., 0.1 to 1 μM, 0.5 to 1 μM, 0.1 to 0.5 μM and the like. Depending on the dye being used, different pH ranges can be used for the complexation reactions. Preferred pH ranges are those wherein the dye species are more reactive towards and metal ions, and form stable colored complexes. These will vary based on the dyes being used.

Any polymeric film may be used in the context of the present invention. In some embodiments, the polymeric film is selected from the group consisting of polyvinyl chloride (PVC), polyvinyl alcohol (PVA), polyvinyl chloride-co-vinyl acetate, polyethylene (PE), polypropylene (PP), polybutene (PB), polymethylpentene, polycarbonates, polyesters, polyacrylates, polyamides and the like. In one preferred embodiment, the polymeric film is polyvinyl chloride (PVC)/polyvinyl alcohol (PVA). Suitable polymeric films are further described, e.g., in U.S. Pat. No. 7,670,843, the contents of which are incorporated by reference herein. The polymer:dye ratio can vary, but typically the solvent to polymer ratios are about 90:10 to 10:90, for example 60:40 or 50:50. The optimal solvent to polymer ratio will depend on the nature of the specific polymer or dye, and can be determined by a person of ordinary skill in the art.

The organic solvent is preferably Bis(2-ethylhexyl)phthalate (DEHP). Other suitable organic solvents are phthalate esters, such as di-isononyl phthalate (DINP), bis(n-butyl)phthalate, butyl benzyl phthalate, diisodecyl phthalate, di-n-octyl phthalate, diisooctyl phthalate, diethyl phthalate, diisobutyl phthalate, di-n-hexyl phthalate, and the like. Citrates may also be used as the organic solvents, e.g., Triethyl citrate (TEC), Acetyl triethyl citrate (ATEC), Tributyl citrate (TBC), Acetyl tributyl citrate (ATBC), Trioctyl citrate (TOC), Acetyl trioctyl citrate (ATOC), Trihexyl citrate (THC), Acetyl trihexyl citrate (ATHC), Butyryl trihexyl citrate (BTHC, trihexyl o-butyryl citrate), Trimethyl citrate (TMC), and the like.

A wide variety of metals can be detected by the methods and kit of the present invention. Thus, the methods of the presence invention are suitable for the detection of heavy metals such as, but not limited to: Cadmium (Cd), Nickel (Ni), Cobalt (Co), Copper (Cu), Arsenic (As), Beryllium (Be), Lead (Pb), Mercury (Hg), Aluminum (Al), Antimony (Sb), Iron (Fe), Manganese (Mn), Palladium (Pd), Platinum (Pt), Selenium (Se), Silver (Ag), Tin (Sn), Uranium (U) Vanadium (V), and Zinc (Zn), rare earth metals such as Scandium (Sc), Yttrium (Y), Lanthanum (La), Cerium (Ce), Praseodymium (Pr), Neodymium (Nd), Promethium (Pr), Samarium (Sm), Europium (Eu), Gadolinium (Gd), Terbium (Tb), Dysprosium (Dy), Holmium (Ho), Erbium (Er), Thulium (Tm), Ytterbium (Yb), and Lutetium (Lu). The metals to be detected can be metal ions, metals in elemental form, metal oxides, or combinations thereof. In one embodiment, the metals can be detected in ionic form (e.g., metal salts). In another embodiment, the metals can be detected in their neutral (elemental) form. In another embodiment, the metals can be detected in the form of metal oxides. Each possibility represents a separate embodiment of the present invention.

Detection of the metals in accordance with the principles of the present invention may be performed by visual inspection and/or spectral analysis, with each possibility representing a separate embodiment of the present invention. Visual analysis may be performed by comparing the color of the polymeric film before and after contact with the aqueous medium suspected of comprising metals. Detection of color changes upon contact with the aqueous medium may be used to detect metals in the aqueous medium. If desired, a comparison color chart may be used in the context of the present invention (e.g., as part of a kit) to allow the user to compare the color of the polymeric film to colors known to be associated with dye-metal complexes of various metals.

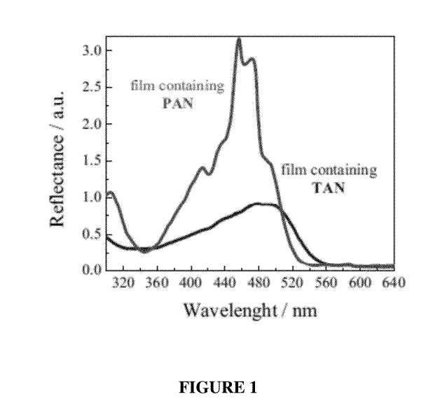

Spectral analysis may be performed by any means known to a person of skill in the art. In one preferred embodiment, spectral analysis comprises measuring the reflectance spectra of the polymeric film. The term “reflectance”, as used herein, refers to a combination of total reflectance (diffused plus specular) and absorption, as further described in the experimental section hereinbelow. Advantageously, the measuring step may be performed at a wavelength at which the dye-metal complex reflects, and wherein the dye has a small or no reflectance. For example, a UV-Vis spectrometer may be used for this purpose. In the case of TAN or PAN dyes, it has been found that PAN in polyvinyl chloride (PVC)/polyvinyl alcohol (PVA) films reflects at 460 nm and TAN at 500 nm. The complexes of each dye with heavy metals possesses reflectance at ca. 600 nm, where the dyes themselves have small reflectance (FIG. 1). Therefore, nearby this wavelength the peaks of the complexes can easily be resolved from those of the dyes themselves. Any spectrometer known in the art may be used for the purpose of the present invention.

The polymeric film used in the present invention may be prepared by any method known to a person of skill in the art. In one preferred embodiment, the polymeric film is prepared by polymerizing monomers in the presence of the organic solvent and the organic dye as described herein. The polymeric film the present invention can be synthesized using standard solution methods (see, for example, Bodanszky, M., Principles of Peptide Synthesis, Springer-Verlag, 1984, the content of which is hereby incorporated by reference in its entirety). Alternatively, the peptide can by produced via solid-phase synthesis (see, for example, Merrifield, 1963, J. Am. Chem. Soc. 85:2149-2154, the contents of which are hereby incorporated by reference in their entirety). Examples of solid phase peptide synthesis methods include, but are not limited to, the BOC method, which utilizes tert-butyloxcarbonyl as the α-amino protecting group, and the FMOC method, which utilizes 9-fluorenylmethyloxcarbonyl to protect the α-amino of the amino acid residues, both methods are well-known by those of skill in the art.

As used herein and in the appended claims the singular forms “a”, “an,” and “the” include plural references unless the content clearly dictates otherwise.

The principles of the present invention are demonstrated by means of the following non-limitative examples.

### EXPERIMENTAL DETAILS SECTION

### Example 1

### Experimental Conditions

Dyes and Ion Solutions

Two azo-dyes complexants were used: (1-(2-pyridylazo)-2-naphthol (PAN), obtained from Mercury, and 1-(2-triazolylazo)-2-naphtol (TAN) from Sigma-Aldrich.

The metal ion concentrations were prepared from certified solutions (Sigma-Aldrich). 1 mM sodium dodecyl sulfate (Merck Schuchardt OHG) was added as an anionic surfactant. Unless otherwise noted, the pH in the TAN solutions was 6.15, adjusted with MES hydrate buffer (minimum 99.5% titration) from Sigma-Aldrich. The pH in the PAN solutions was 10.6, adjusted with ammonia Suparpur (Merck Schuchardt OHG).

Polymeric Film

The polymeric film was designed as follows: The skeleton used for obtaining a solid film comprises 90% polyvinyl chloride (PVC)/10% polyvinyl alcohol (PVA). Bis(2-ethylhexyl)phthalate (Sigma) was added as an organic solvent entrapped in the skeleton at a solvent to polymer ratio of 60:40. Without wishing to be bound by any particular theory of mechanism of action, it is believed that the role of this solvent is to change the dielectric properties of the polymeric film and the diffusivity of the cations, such that it may extract and accommodate the metal complexes. In order to facilitate the absorption of nano-complexes and ions from water, small quantities of the azo dyes were added to the solution. The solution was placed on a flat glass surface and polymerized under THF saturated atmosphere at 60° C. The dry polymeric matrix was sliced to squares and gently separated from the glass support. The mean thickness of the obtained polymeric films was ca. 0.2 mm.

Spectral Analysis

The resulted polymeric films were spectrally inspected using a double beam UV-Vis spectrometer (Evolution 300, Thermo Fisher Scientific Inc., USA), equipped with a Xe flash-lamp, providing 0.5 nm spectral resolution. Surface diffused reflectance was collected using an integrating sphere (DRA-EV-300, Thermo Scientific Inc., USA). The examined film was placed on top of the integrating sphere's window and covered by a white sintered PTFE sheet (Fluorilon 99W). Therefore, a part of the light transmitted through the examined film was reflected back into the sphere and contributed to the signal. This allowed for higher and more significant signals. Accordingly, the spectra reported herein, which are denoted as “reflectance”, for simplicity, are a combination of total reflectance (diffused plus specular) and absorption.

Particle Characterization

Particle size measurements were performed using a zeta potential particle seizing system (Nicomp 380 ZLS, Santa Barbara, Calif.). Imaging of the dye and complex particulates was performed using a Transmittance Electron Microscope (FEI Tecnai G² T20 S-Twin).

Measuring Procedure

The tested water was double deionized water spiked with heavy metals at the desired concentrations. The desired solution pH was adjusted by addition of NH₄OH or HCl. The pH was rechecked at the end of the measurement and found unchanged.

The heavy metals tested included Ni, Cd and Co ions. Azo dyes solutions in acetone were injected in the tested water at the desired concentration (see the optimization procedure in Example 2—Results). The polymeric films were immersed in the tested water and gently stirred for the desired time (as indicated in Example 2). At the end, the polymeric films were visually inspected, photographed and their spectra were measured.

### Example 2

### Results and Discussion

The reflectance spectra of the polymeric films, before their exposure to heavy metals, are shown in FIG. 1. The PAN in this film reflects at 460 nm and the TAN reflects at 500 nm. Fortunately, the complexes of both dyes with heavy metals possess reflectance at ca. 600 nm, where the dyes themselves have a small reflectance. Therefore, nearby this wavelength the peaks of the complexes can easily be resolved from those of the dyes.

Effect of the Dye Concentration in the Polymeric Film

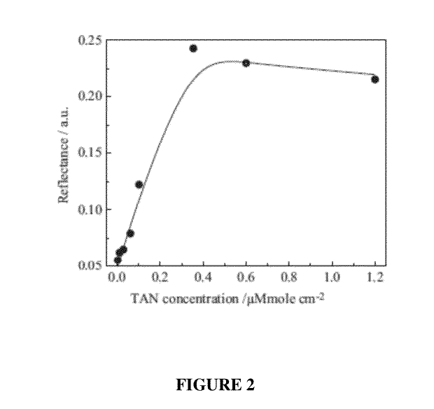

The dye concentration affects the sensitivity of the polymeric film to heavy metals. For example, in FIG. 2 the reflectance of the film is presented as a function of TAN surface concentration. The measurement was taken at 598 nm, at the reflectance band of the Ni-TAN complex. In all these measurements the sampling time and the Ni ion concentrations were kept constant (90 min, 5 ppm). The reflectance increases with the concentration of the organic dye up to a value of ca. 0.4 μmole cm⁻² where the signal reaches a plateau. At the concentration of 0.3 μmole cm⁻² the sensitivity was high and below the plateau. Therefore this concentration was used in all following experiments.

Generation of Nano-Complexes in Solution

Both the organic dyes and their complexes with heavy metals are not soluble in water. Therefore, when the organic dye (dissolved in acetone) is injected into an aqueous solution containing heavy metals, it immediately forms nano-particles of the dye, which then react and form nano-particles of the dye-metal complex.

Characterization of both the dye and the complex nano-particles was conducted using TEM and particle seizer. It was found that injection of TAN into nickel ion solution results in the generation of particles of bimodal size distribution: One peak is centred at 100 nm and the second at 600 nm. Similar results were obtained for injection of PAN into cadmium ion solutions. In this case, too, the first peak was centred at 100 nm, however, the second one was observed at a much larger diameter of 1500 nm.

The polymeric film used herein is rich in organic solvent and can readily absorb and dissolve both dye and complex nano-particles. In these experiments the dye is not only injected in the water solution, but it is also introduced (dissolved) into the polymeric film. Analysis is based on observing the complexes present in the film. Without wishing to be bound by any particular theory or mechanism of action, these might have three sources: (1) The nano-complexes created in the solution, which are slowly absorbed and dissolved into the film. This mechanism is referred hereinafter as complex extraction. (2) Metal ions from the solution that penetrate into the polymeric film and react with the dye in the film (most probably, the complexation takes place at the film's surface). (3) Complex molecules which decompose on the film surface and transfer their ion to the film.

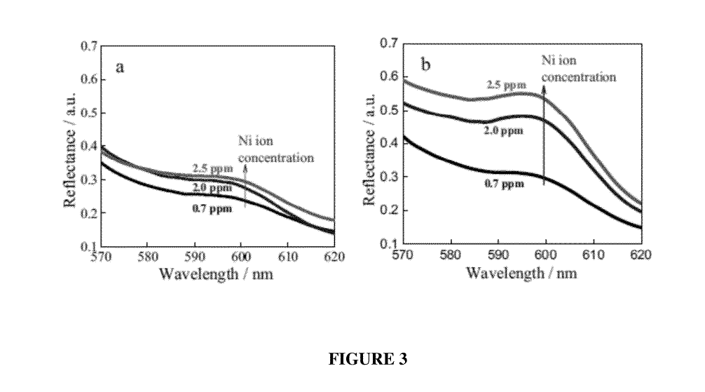

The first contribution, complex extraction, appears to be predominant. The major contribution of complex extraction to the coloration of the polymeric film is exemplified in FIG. 3. The film reflectance spectra in the region corresponding to the Ni-TAN complex, is shown for a series of Ni ion concentrations. When only trace amount of TAN is added to the examined water, (FIG. 3a) the complex reflectance only slightly increases with Ni ion concentration. This minor increase is mainly related to complexation in the film after penetration of Ni ions. However, when a considerable amount of TAN is injected into the water, (FIG. 3b) a large increase in the Ni-TAN reflectance is observed. This major contribution to the film's reflectance is related to complex nano-particles that are absorbed into the film.

Kinetics of Complex Formation and Accumulation

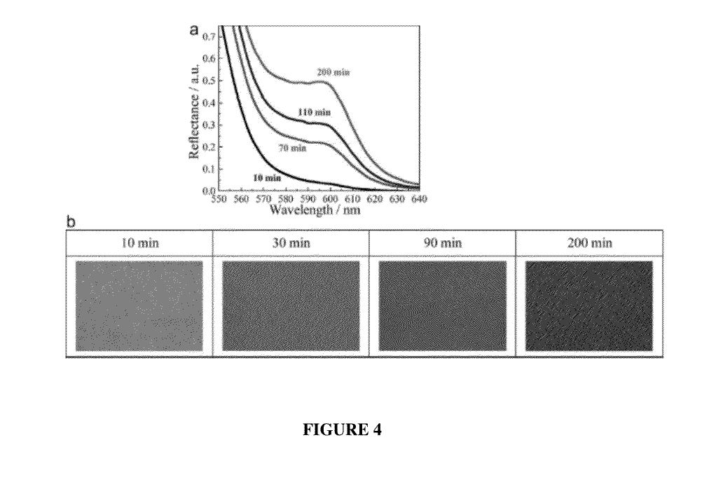

Once the polymeric film is immersed in the examined water, the two main complex accumulation processes start (complex extraction and in-film complexation). The kinetics are shown in FIG. 4a, for a TAN containing film, exposed to 2.5 ppm Ni ion solution. A monotonic increase in the reflectance with exposure time is observed. The results can also be monitored by the naked eye (FIG. 4b).

Effect of Dye Concentration Upon Sensitivity

As shown, the major contribution to the measured reflection is due to metal-dye complexes formed in the solution and then absorbed/dissolved into the polymeric film. These are complex nanoparticles, which are accommodated into the film due to its special composition. Next, the effect of the dye concentration in the tested solution upon method sensitivity was investigated.

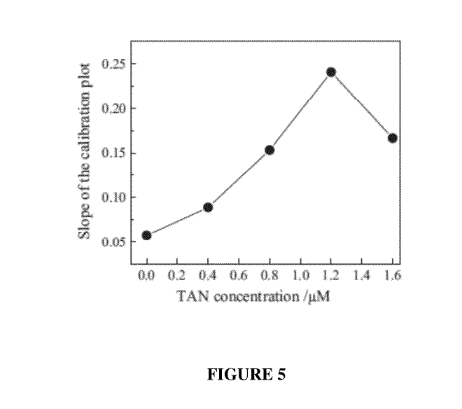

The experimental results indicate that although linear calibration plots can be obtained at all dye concentrations, the sensitivity to heavy metals (namely the slope of the calibration plot) strongly depends on the dye concentration. For example, FIG. 5 presents the slope of the calibration plots for detection of Ni ions. The slope increases with dye concentration. However, after a certain concentration (ca. 1.2 μM) the coloration of the films becomes very intense so that the absorption is no longer dependent on the metal ion concentration and the slope slightly descends.

Effect of pH

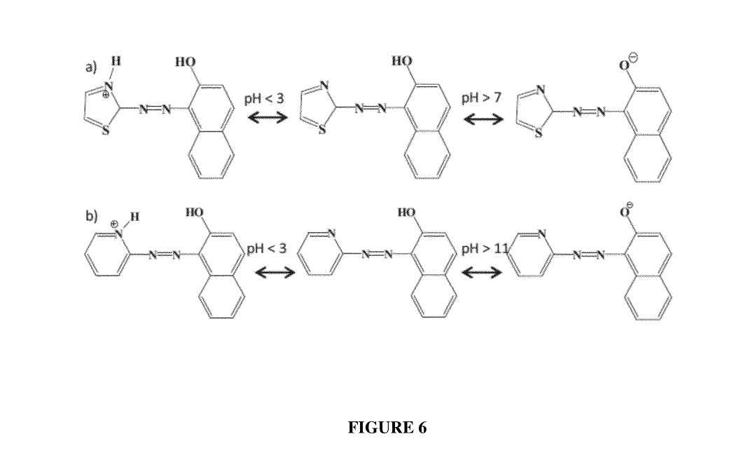

The sorption of the metal-dye complex into the polymeric film is also affected by the pH of the solution. It is evident that film coloration increases as the pH increases. This effect is directly related to the different azo dye species stabilized at various pH ranges. In acidic solutions (pH<3), the dye molecules are protonated (H₂TAN)⁺, for example, while the neutral form (HTAN) is stable in the pH range 4-6. The monobasic species are predominant at pH>7 for TAN and pH>11 for PAN. Both the acidic and basic forms are water soluble. The monobasic species are more reactive towards metal ions and forms stable colored complexes. The corresponding TAN and PAN species are shown in FIG. 6.

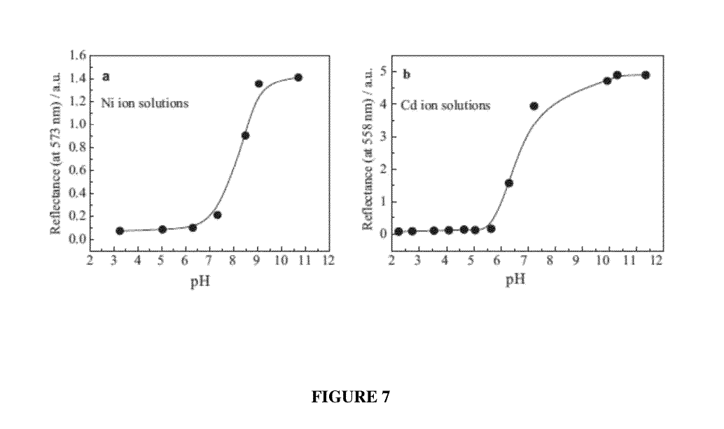

The above scheme implies that the formation of the nickel azo dye complex should be preferred in basic solutions. In order to test this effect, the PAN and TAN based films were submerged into 2.5 ppm heavy metal ion solutions (Ni and Cd, separately) for 70 min, at a series of pH conditions. For example, the resulted PAN based film reflectance is shown in FIG. 7. The measurements were performed at the wavelengths corresponds to the PAN complex of the relevant metal ion. Indeed, the reflectance increases with the pH. Both dyes PAN and TAN are affected by the solution pH in a similar way, but there are differences in the pH at which the increase in the reflection starts. Thus, the pH regime may be used for achieving selectivity for a certain metal in the solution. However, the highest signals are expected at high pH environments, where selectivity is compromised.

It was shown that, while selectivity to a given metal can be achieved by selecting the proper dye and pH values, at very high pH both PAN and TAN form complexes with heavy metals. To test which complexant performs better at high pH values, an analysis of Cd ions at a series of concentrations was carried out using both complexants. The LOD obtained with PAN in the polymeric film (and solution) was lower than that obtained with TAN.

Calibration Plots for Ni Ions

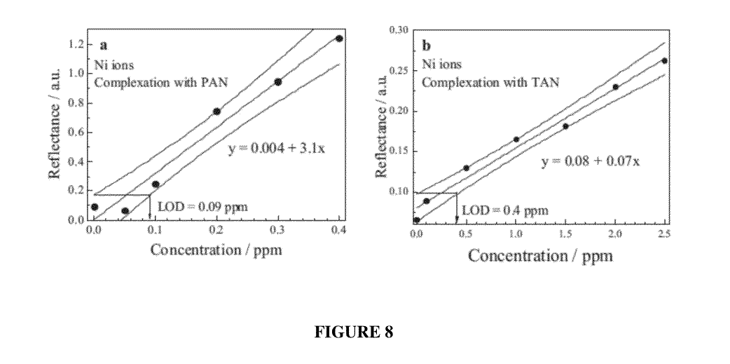

The polymeric films provided linear calibration plots for heavy metals in the sub-ppm range. An example of such a plot is shown in FIG. 8, for Ni ions in aqueous solutions detected using PAN and TAN embedded polymeric film. The calculated 95% confidence interval detection limit was 90 ppb (for PAN) and 400 ppb (for TAN). The slope of the calibration plot for the PAN was much higher than for TAN. Therefore, in this experiment, PAN is shown to be a better complexant for analysis of Ni ions.

Visual Assessment of Ni Ion Concentration

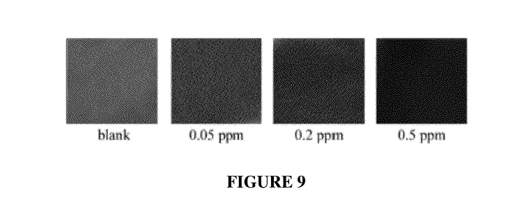

The color change of the polymeric film exposed to heavy metal solutions can be observed by the naked eye and can be used for concentration assessment. Examples of films exposed to several Ni ion concentrations, in the sub-ppm range, are shown in FIG. 9.

Calibration Plots for Cd Ions

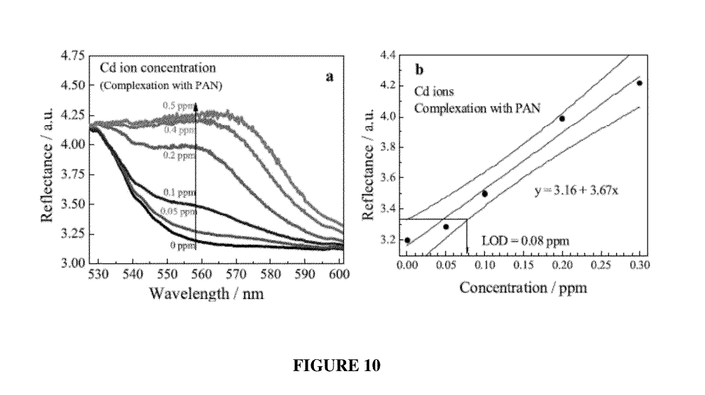

Similar experiments have been carried out for detection of Cd ions. Also for this metal the PAN complexant provided better results. The PAN concentration in the film was 1.1 μmole cm⁻² and its concentration in the tested solution was 1.2 μM. The pH was adjusted to 10.3 with ammonium hydroxide and the integration time was 70 min. The corresponding spectra and the resulted calibration plot are shown in FIG. 10 The 95% confidence interval based LOD was 77 ppb.

Calibration Plots for Co Ions

Similar experiments have been carried out for detection of Co ions, using the PAN and TAN complexants. In this case the PAN concentration in the film was 1.13 μmole cm⁻² and its concentration in the tested solution was 1.2 μM. The pH was 10.6 and the integration time was 70 min. The best calibration plots were obtained at 638 nm (with PAN) and at 573 nm (with TAN).

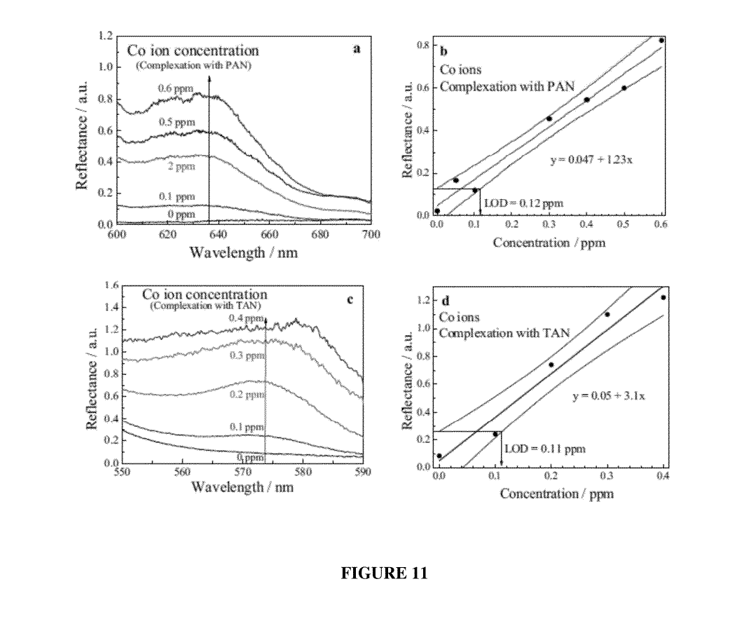

The thus obtained spectra and the resulted calibration plots are shown in FIG. 11 The 95% confidence interval based LOD was 120 ppb, when using PAN, and 110 ppb, when using TAN. In the latter case, the slope of the calibration plot was higher.

### CONCLUSIONS

Complexation of heavy metals with organic complexants is a well-known analytical technique. However, since both the complexants and their products are insoluble in water, direct analysis is problematic and suffers from intrinsic inaccuracy related to the resulting suspension of nano-particles. The present invention suggest for the first time using a specially designed polymeric film, which can absorb and dissolve the particulate complexes and thus allow for more accurate spectral measurements. As demonstrated herein, it has been found that a polymeric film of the required characteristics can be synthesized and that it works well for sampling of particulate dye-metal complexes and for quantitative measurements of heavy metals in water. The current sensitivity is in the tens ppb range, which can be further optimized by optimizing the film thickness, its chemical composition and even its preparation mode. Detailed kinetic investigation revealed that the major mechanism is absorption of complexes from the solution into the polymeric film. A parallel minor mechanism of decomposition of the complex on the polymeric surface and transferring the metal ion into the film also takes place (data not shown).

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

### REFERENCES

[1] N. F. Savage, M. Diallo and N. Savage, Nanotechnology Applications for Clean Water, William Andrew, 2008 p. 418-424.
[2] K. Krishnankutty and D. K. Babu, Metal Complexes of 2-(2-Thiazolylazo)-1,3-Dicarbonyls, Indian Chemical Society, Calcutta, INDE, 1996.
[3] M. M. Omar and G. G. Mohamed, Potentiometric, spectroscopic and thermal studies on the metal chelates of 1-(2-thiazolylazo)-2-naphthalenol, Spectrochimica Acta Part A: Molecular and Biomolecular Spectroscopy, 61 (2005) 929-936.
[4] C. R. T. Tarley, W. N. L. dos Santos, C. M. dos Santos, M. A. Z. Arruda and S. L. C. Ferreira, Factorial Design and Doehlert Matrix in Optimization of Flow Kit for Preconcentration of Copper on Polyurethane Foam Loaded with 4-(2-Pyridylazo)-resorcinol, Analytical Letters, 37 (2004) 1437-1455.
[5] Y. Sumi and T. Suzuki, Recent advances in the histochemical staining of heavy metals by means of chelating agents, with special reference to cadmium, Microscopy Research and Technique, 56 (2002) 332-340.
[6] H. Eskandari and A. Ghanbari Saghseloo, Second and First-Derivative Spectrophotometry for Efficient Simultaneous and Individual Determination of Palladium and Cobalt Using 1-(2-Pyridylazo)-2-naphthol in Sodium Dodecylsulfate Micellar Media, Analytical Sciences, 19 (2003) 1513-1518.
[7] L. Cornejo-Ponce, P. Peralta-Zamora and M. I. M. S. Bueno, Pre-concentration of rare earths using silica gel loaded with 1-(2-pyridylazo)-2-naphthol (PAN) and determination by energy dispersive X-ray fluorescence, Talanta, 46 (1998) 1371-1378.
[8] R. L. Sharma, H. B. Singh and M. Satake, Spectrophotometric and high-order derivative spectrophotometric study of the indium-1-(2-pyridylazo)-2-naphthol complex in the aqueous phase, Analyst, 111 (1986) 551-553.
[9] A. M. Starvin and T. P. Rao, Removal and recovery of mercury(II) from hazardous wastes using 1-(2-thiazolylazo)-2-naphthol functionalized activated carbon as solid phase extractant, Journal of Hazardous Materials, 113 (2004) 75-79.
[10] M. Tuzen, K. Parlar and M. Soylak, Enrichment/separation of cadmium(II) and lead(II) in environmental samples by solid phase extraction, Journal of Hazardous Materials, 121 (2005) 79-87.
[11] I. Narin, M. Soylak, K. Kayakirilmaz, L. Elci and M. Dogan, Preparation of a Chelating Resin by Immobilizing 1-(2-Pyridylazo) 2-Naphtol on Amberlite XAD-16 and Its Application of Solid Phase Extraction of Ni(II), Cd(II), Co(II), Cu(II), Pb(II), and Cr(III) in Natural Water Samples, Analytical Letters, 36 (2003) 641-658.
[12] M. Saeed, S. Bajwa, M. Ansari and R. Ahmed, Investigation of the removal of lead by adsorption onto 1-(2-thiazolylazo)-2-naphthol (TAN) imbedded polyurethane foam from aqueous solution Chinese chemical society taipei 54 (2007) 173-183.
[13] S. L. C. Ferreira, J. B. de Andrade, M. d. G. A. Korn, M. d. G. Pereira, V. A. Lemos, W. N. L. d. Santos, F. d. M. Rodrigues, A. S. Souza, H. S. Ferreira and E. G. P. da Silva, Review of procedures involving separation and preconcentration for the determination of cadmium using spectrometric techniques, Journal of Hazardous Materials, 145 (2007) 358-367.
[14] V. A. Lemos, E. S. Santos, M. S. Santos and R. T. Yamaki, Thiazolylazo dyes and their application in analytical methods, Microchimica Acta, 158 (2007) 189-204.
[15] P. P. Hankare and S. S. Chavan, Synthesis and characterization of manganese(II), cobalt(II), nickel(II), copper(II) and zinc(II) complexes with 2-[2′-hydroxy salicylidene 5′-(4″-phenyl, 2″-thiazolylazo)]benzoic acid and 2-[2′-hydroxy salicylidene 5′-(4″-phenyl, 2″-thiazolylazo)]phenol, India National Science Academy, New Delhi, INDE, 2003.
[16] Z. Li, G. Yu, J. Song, Q. Wang, M. Liu, Y. Yang, Study on the determination of heavy metals in water samples with ultrasound-assisted dispersive liquid-liquid microextraction prior to FAAS, Water Sci. Technol. 67.2, 2013, 247-253.

What is claimed is:

1. A method for detecting metals in an aqueous medium, the method comprising the steps of:
(a) contacting a test aqueous medium comprising an organic dye with a polymeric film comprising an organic solvent and said organic dye; and
(b) detecting the presence of metals in said aqueous medium by visual inspection and/or by spectral analysis of the polymeric film.

2. The method according to claim 1, wherein said polymeric film comprises polyvinyl chloride (PVC)/polyvinyl alcohol (PVA).

3. The method according to claim 1, wherein the organic dye is an azo dye.

4. The method according to claim 3, wherein the azo dye is selected from the group consisting of 1-(2-thiazolylazo)-2-naphthol (TAN) and (1-(2-pyridylazo)-2-naphthol (PAN).

5. The method according to claim 1, wherein the organic solvent is Bis(2-ethylhexyl)phthalate.

6. The method according to claim 1, wherein the aqueous medium comprises heavy metals selected from the group consisting of cadmium (Cd), nickel (Ni), cobalt (Co), copper (Cu), including their ionized forms, i.e., salts or oxides, and combinations thereof.

7. The method according to claim 1, wherein said dye complexes with said metals so as to form a metal-dye complex.

8. The method according to claim 7, wherein said metal-dye complex is formed in the aqueous medium and is absorbed/dissolved into the polymeric film, and/or wherein the metal forms a complex with the dye inside the film or on the film surface.

9. The method according to claim 1, wherein the polymeric film is prepared by polymerizing monomers in the presence of said organic solvent and said organic dye.

10. The method according to claim 1, wherein the polymeric film is provided in the form of a test strip.

11. The method according to claim 1, wherein the contacting step (a) comprises submerging part or all of the polymeric film in the aqueous medium.

12. The method according to claim 1, wherein the detection step (b) comprises comparing the color of the polymeric film before and after contact with said aqueous medium, wherein a change in the color of said polymeric film is indicative of the presence of metals in said aqueous medium.

13. The method according to claim 1, wherein the detection step (b) comprises comparing the reflectance spectra of the polymeric film before and after contact with said aqueous medium, wherein a change in reflectance spectra of said polymeric film is indicative of the presence of metals in said aqueous medium.

14. The method according to claim 13, wherein said reflectance spectra is measured at a wavelength at which the dye-metal complex reflects, and wherein the dye has a small or no reflectance.

15. The method according to claim 1, further comprising the step of adding said organic dye to said aqueous medium prior to or simultaneously with step (a).

16. The method according to claim 15, wherein the organic dye is dissolved in a water-miscible organic solvent prior to addition to said aqueous medium.

17. A device for detecting metals in an aqueous medium, the device comprising a polymeric film comprising an organic solvent and an organic dye wherein the polymeric film is selected from polyvinyl chloride (PVC), polyvinyl alcohol (PVA) and combinations and copolymers thereof the organic solvent is Bis(2-ethylhexyl)phthalate, and the organic dye is an azo dye selected from the group consisting of 1-(2-thiazolylazo)-2-naphthol (TAN), (1-(2-pyridylazo)-2-naphthol (PAN), and combinations thereof.

18. A kit for detecting metals in an aqueous medium, the kit comprising
(a) a polymeric film comprising an organic solvent and an organic dye; and
(b) a composition comprising said organic dye;
wherein the presence of metals is detected by adding said organic dye composition to said aqueous medium followed by contacting said aqueous medium with said polymeric film, and detecting the presence of metals in said aqueous medium by visual inspection and/or by spectral analysis of the polymeric film.

19. A system for detecting metals in an aqueous medium, the system comprising:
(a) a polymeric film comprising an organic solvent and an organic dye; and
(b) an aqueous medium comprising said organic dye;
wherein the presence of metals is detected by contacting said aqueous medium with said polymeric film, and detecting the presence of metals in said aqueous medium by visual inspection and/or by spectral analysis of the polymeric film.

20. The device according to claim 17, the kit according to claim 18 or the system according to claim 19, wherein said polymeric film comprises polyvinyl chloride (PVC)/polyvinyl alcohol (PVA).

21. The device according to claim 17, the kit according to claim 18 or the system according to claim 19, wherein the organic dye is an azo dye.

22. The device, kit or system according to claim 21, wherein the azo dye is selected from the group consisting of 1-(2-thiazolylazo)-2-naphthol (TAN) and (1-(2-pyridylazo)-2-naphthol (PAN).

23. The device according to claim 17, the kit according to claim 18 or the system according to claim 19, wherein the organic solvent is Bis(2-ethylhexyl)phthalate.

24. The device according to claim 17, the kit according to claim 18 or the system according to claim 19, wherein the aqueous medium comprises heavy metals selected from the group consisting of cadmium (Cd), nickel (Ni), cobalt (Co), copper (Cu), including their ionized forms, i.e., salts or oxides, and combinations thereof.

25. The device according to claim 17, the kit according to claim 18 or the system according to claim 19, wherein the polymeric film is provided in the form of a test strip.

26. The kit according to claim 18, further comprising a chart indicating known colors of dye-metal complexes.
